(19) United States
(12) Patent Application Publication
(10) Pub. No.: US 2009/0248106 A1
(43) Pub. Date: Oct. 1, 2009

(54) Optical wireless system for electrophysiological stimulation

(76) Inventor: Michael D. Black, Palo Alto, CA (US)

Correspondence Address: LUMEN PATENT FIRM, 350 Cambridge, Suite 100, PALO ALTO, CA, 94306, US

(21) Appl. No.: 12/383,577

(22) Filed: Mar. 24, 2009

Related U.S. Application Data
(60) Provisional application No. 61/070,705, filed on Mar. 24, 2008.
(60) Provisional application No. 61/209,525, filed on Mar. 5, 2009.

(51) Int. Cl. A61N 1/378; A61N 1/34; A61N 1/02

(52) U.S. Cl. 607/33; 607/46; 607/61

(57) ABSTRACT

Optical-based wireless systems for electrophysiological stimulation are provided. One or more small implantable devices, referred to as trigger pods, receives infrared light transmitted from an optical transmitter and converts the light into electrical energy, which is then used to generate electrical impulses. The impulses are used for biomedical applications, such as cardiac pacing and neurostimulation for pain relief. Because the trigger pods are battery-less and rely solely on the incident optical signals for power, they can be highly miniaturized for ease of deployment into the body of a patient. The optical signals can also be used for data/signal transmission in addition to power transmission for greater control of the electrical stimulation. Systems having optical fibers and implantable transmitters are also provided.

### CROSS-REFERENCE TO RELATED APPLICATIONS

This application claims priority from U.S. Provisional Patent Application 61/070,705, Docket No. MDB-101/PROV, titled “Wireless-based Cardiac Pacing” and filed Mar. 24, 2008, which is incorporated herein by reference. This application also claims priority from US Provisional Patent Application Docket No. MDB-103/PROV, titled “Electrophysiological Stimulation System Using Optical Signals” and filed Mar. 5, 2009, which is incorporated herein by reference.

### FIELD OF THE INVENTION

The invention relates generally to electrophysiological stimulation. More particularly, the present invention relates to electrophysiological stimulation with a wireless system using optical-based communication and power transmission.

### BACKGROUND

An electrophysiological stimulation device is a medical device that uses electrical impulses delivered by electrodes contacting muscles or nerves to regulate or stimulate their function. Applications for electrophysiological stimulation include artificial cardiac pacemakers and devices designed for neurostimulation. Many existing electrophysiological stimulation devices rely on a wired architecture where implanted leads are wired to a central device. For example, current designs for pacemakers use intravenously inserted pacing leads attached internally within the chambers of the heart and wires to link the leads to the pacemaker, which determines when electrical pulses are delivered. In another example, existing Implantable neurostimulator devices, incorporating pulse generators, provide electrical stimulation through wired leads implanted near the central nervous system, i.e. (the brain or spinal cord) or an affected peripheral nerve.

Wired electrophysiological stimulation systems face many difficulties with performance and infection to the patient. Under a wired architecture, there is limited flexibility in routing of the electric leads, especially to multiple locations. Wired intravenous cardiac devices for example, are especially problematic in those with limited body surface areas i.e. children and young adults. Thrombosis of the venous conduits and downstream embolization are recognized complications. Undue tension on vital structures can occur during somatic growth and the removal of wired devices is currently fraught with significant morbidity and mortality. Wired architecture is also limited in scalability. In addition, the large size of the implanted central device or leads can provide discomfort to the patient. When the central device is externally located and wired to the implanted leads, the probability of infection at the wire/skin interface remains high.

Wireless electrophysiological stimulation systems have been developed to overcome some of the above disadvantages of wired systems. Some wireless and leadless electrophysiological stimulation systems have been developed using RF/microwave and also ultrasonic acoustic technology. These devices use high-energy radio waves or ultrasonic waves from an external power source for wireless communication and also to recharge the battery in the implanted devices, or else to convert the incident RF/ultrasound energy directly into electrical power.

Though existing wireless electrophysiological systems overcome some of the disadvantages of wired systems, there remain many difficulties in these wireless systems. For implanted devices relying solely on internal batteries to operate, the longevity and power of the device is limited. Frequent surgical procedures would be required for higher battery usage applications. Though RF devices need not be surgically removed to recharge, they also face difficulties with electromagnetic interference. In addition, RF systems typically still require rechargeable batteries in the implanted devices to temporarily store charge in the devices. The wireless electrophysiological stimulation systems based on ultrasound technology can have safety issues related to prolonged exposure of biological tissues to ultrasound acoustic energy, and can be constrained by low transmission efficiencies, especially in internal body cavities. Adverse changes in cellular ultrastructure (thermal or cavitation damage) have been previously demonstrated.

The presence of a battery in an implantable device limits the miniaturization of the device. The large size of battery-powered (either rechargeable or non-rechargeable) devices often causes discomfort to the patient and increases the risk of nerve or tissue damage from external mechanical shocks. Furthermore, large devices face difficulties in deployment inside of the body, and raise the probability of infection.

The present invention addresses at least the difficult problems of electrophysiological stimulation and advances the art with a wireless system for providing electrical stimulation.

### SUMMARY OF THE INVENTION

The present invention is directed to optical-based wireless devices, systems, and methods for electrophysiological stimulation. In a preferred embodiment, an implantable device, referred to as a trigger pod, is provided for delivering electrophysiological stimulation to a subject. The device includes a micro-power panel for receiving a wirelessly transmitted optical signal, wherein the optical signal includes infrared light, and wherein the micro-power panel converts the infrared light into electrical energy; an electronic circuit for generating electrical impulses, wherein the electronic circuit is powered by the electrical energy converted by the micro-power panel; and one or more electrodes, wherein the electrical impulses generated by the electronic circuit are delivered to the subject through the one or more electrodes, and wherein the device is implantable near a muscle, a tissue, or a nerve internal to the subject. Preferably, the device does not include a battery. In an embodiment, the device also includes a lens to focus the incident optical signal onto the micro-power panel.

In an embodiment, the micro-power panel includes one or more photodiodes and the optical signal received by the micro-power panel is a nearly collimated optical beam. In another embodiment, the micro-power panel receives a second optical signal for data transmission, wherein the second optical signal includes a modulated optical beam and directs the electronic circuit to control the intensity, the duration, the timing, or any combination thereof of the electrical impulses. Alternatively, the optical signal includes a modulated beam for both power and data transmission. In a preferred embodiment, the trigger pod is less than approximately 7 mm in width. In certain embodiments, the trigger pod includes an energy-harvesting module, wherein the energy-harvesting module uses vibrational or thermal energy to partially power the device.

The present invention is also directed to a wireless system for providing electrophysiological stimulation to a subject. The system includes an optical transmitter for transmitting optical signals and one or more implantable trigger pods, wherein each of the trigger pods includes: a micro-power panel for receiving the optical signals transmitted by the optical transmitter, wherein the micro-power panel converts the optical signal into electrical energy; an electronic circuit for generating electrical impulses, wherein the electronic circuit is powered by the electrical energy converted by the micro-power panel; and one or more electrodes, wherein the electrical impulses generated by the electronic circuit are delivered to the subject through the one or more electrodes, wherein the one or more trigger pods are implanted near a muscle (skeletal, smooth or cardiac), a tissue, or a nerve internal to the subject, and wherein the one or more trigger pods are wirelessly connected to the optical transmitter. In a preferred embodiment, the implantable trigger pods are battery-less.

In an embodiment, the optical transmitter includes a laser diode or a light-emitting diode for producing optical signals. The optical transmitter can also include one or more mirrors to direct the optical signals from the optical transmitter to the trigger pods. In an embodiment, the mirrors are rotatable. The optical transmitter can be implanted in the body of the subject or can be external to the subject. In another embodiment, the optical transmitter transmits a second optical signal, wherein the micro-power panel of one of the trigger pods receives the second optical signal, and wherein the second optical signal directs the electronic circuit of the same trigger pod to control the intensity, the duration, the timing, or any combination thereof of the electrical impulses.

In an embodiment, the system includes one or more optical fibers, wherein the optical transmitter transmits the optical signals to the trigger pods through the optical fibers. The optical fibers can be implanted in the body of the subject or can be located external to the subject. In an embodiment, the system includes a multi-furcated fused fiber bundle, wherein optical signals are delivered through the legs of the multi-furcated fused fiber bundle to the trigger pods. In an embodiment, an optical fiber has one or more controlled leakage locations, wherein the optical signals exit the optical fiber through the leakage locations and are transmitted to the trigger pods. In an embodiment, the system includes multiple optical transmitters that are communicatively connected.

Another embodiment of the present invention is directed to a method of providing electrophysiological stimulation to a subject. The method includes (1) providing an optical transmitter for transmitting optical signals; (2) implanting one or more trigger pods near a muscle (skeletal, smooth or cardiac), a tissue, or a nerve of the subject, wherein each of the trigger pods includes a micro-power panel for receiving the optical signals transmitted by the optical transmitter, wherein the micro-power panel converts the optical signal into electrical energy, an electronic circuit for generating electrical impulses, wherein the electronic circuit is powered by the electrical energy converted by the micro-power panel, and one or more electrodes, wherein the electrical impulses generated by the electronic circuit are delivered to the subject through the one or more electrodes; and (3) directing the optical transmitter to transmit said optical signals to the trigger pods, whereby the electrical impulses provide electrophysiological stimulation to the muscle, the tissue, or the nerve of the subject.

Another embodiment of the method further includes directing the optical transmitter to transmit a second optical signal to the trigger pods, wherein the micro-power panel of one of the trigger pods receives the second optical signal, and wherein the second optical signal directs the electronic circuit of the same trigger pod to control the intensity, the duration, the timing, or any combination thereof of the electrical impulses delivered by the same trigger pod.

In a preferred embodiment, at least one of the trigger pods is implanted near the heart of the subject, wherein the electrical impulses delivered by the same trigger pod are for treating dyrrhythmias. In another embodiment, at least one of the trigger pods is implanted near one of the nerves of the subject, wherein the electrical impulses delivered by the same trigger pods are for mimicking or blocking neurotransmission, i.e. pain relief to the subject.

### BRIEF DESCRIPTION OF THE FIGURES

The present invention together with its objectives and advantages will be understood by reading the following description in conjunction with the drawings, in which:

FIG. 1 shows an example of an optical wireless system for electrophysiological stimulation according to the present invention.

FIG. 2 shows an example implantable trigger pod according to the present invention.

FIG. 3 shows an example optical transmitter according to the present invention.

FIG. 4 shows an example system having multiple trigger pods according to the present invention.

FIGS. 5A-B show example mirror configurations for an embodiment of an optical transmitter according to the present invention.

FIG. 6 shows an example electrophysiological stimulation system with an implanted optical transmitter and a multi-furcated fiber bundle according to the present invention.

FIG. 7 shows an example electrophysiological stimulation system with a multi-furcated fiber bundle transmitting optical signals through the skin according to the present invention.

FIG. 8 shows an example electrophysiological stimulation system with light leakage in an optical fiber bundle of uniform size according to the present invention.

FIG. 9 shows an example electrophysiological stimulation system having multiple communicatively connected optical transmitters according to the present invention.

FIG. 10 shows an example of an implanted optical transmitter connected with multiple optical fiber bundles according to the present invention.

FIG. 11 shows an example implantable trigger pod receiving multiple optical signals according to the present invention.

FIG. 12 shows an example implantable trigger pod with an energy-harvesting module according to the present invention.

### DETAILED DESCRIPTION OF THE INVENTION

The present invention is directed to wireless optical-based electrophysiological stimulation. In embodiments of the present invention, electrophysiological stimulation can be used to provide relief to people suffering from a variety of conditions, such as neurological disorders, pain relief, spasms, and dysrrhythmia. It is noted that the present invention can be applied for stimulation of any nerve, tissue, or muscle of a human or non-human subject. The following includes a brief description of applications where the present invention can be applied, though it is noted that the present invention is not limited to these applications.

### Deep Brain Stimulation

Electrical stimulation of the deep regions within the brain allows for the treatment of otherwise resistant movement disorders and affective disorders. The stimulation allows for the supporting elements of the brain to release adeonsine triphosphate. Neurohumoral changes can have a positive effect on behavior and emotions.

Deep brain stimulation can be used to treat chronic pain disorders, Parkinson's disease, tremors, dystonia, spasms, depression, and epilepsy. For example, for non-Parkinsonian essential tremor electrical stimulation can be applied to the ventrointermedial nucleus of the thalamus. For dystonia and symptoms related to Parkinson's disease stimulation of the globus pallidus or the subthalamic nucleus is desired.

### Spinal Cord Stimulation

Stimulation of the dorsal column of the spinal cord allows for the altered perception of pain. Stimulation frequently is either epidural or subcutaneous. Frequent applications include failed back syndrome, complex regional pain syndromes and peripheral neuropathies.

### Cranial Nerve Stimulation

Neuralgia or nerve induced pain disorders can be treated by electrical stimulation of the peripheral nerve. Particular examples of pain disorders include occipital neuralgia, trigeminal neuralgia and glossopharyngeal neuralgias.

### Peripheral Nerve Stimulation

Neuralgia or nerve induced pain disorders can be treated by electrical stimulation of the peripheral nerve. Particular examples of pain disorders include median, ulnar and radial neuralgias.

### Skeletal and Smooth Muscle Stimulation

Muscles can be stimulated to produce contraction of the stimulated muscle. Usages could include improved intestinal propulsion and external control of sphincters, such as the anus or the bladder neck.

### Cardiac Stimulation

Normal cardiac muscle stimulation occurs spontaneously via depolarization of the electrically active cells within the myocardium. The remaining cells are activated simultaneously as they remain connected together on a cellular level. When either an abnormal origin or rate of stimulation occurs, artificial electrical stimulation can correct the abnormality. Diseases that can be treated include bradyarrhythmias, tachyarrhythmias, bradytachyarrhythmias, abnormal rhythms originating from either the atria and/or the ventricles.

An embodiment of the present invention is directed to pacemaker applications where electrical stimulation is applied to surface of the heart (epicardial) or within the heart (endocardial). Indirect pacing of the systemic chambers of the heart can be accomplished internally by stimulating the coronary sinus, a venous structure that runs posterior to the systemic pumping chamber. The present invention can by design directly pace selected cardiac chambers, either in an epi- or endocardial configuration or in a combination thereof. Indirect pacing could be achieved if deemed necessary.

The present invention provides a wireless optical-link based system between a main controller with an optical transmitter and remote electrode assemblies, referred to as trigger pods. The optical connection between the transmitter and trigger pods can provide signal transmission, power transport, or both, to the implanted battery-less trigger pods. The present invention can be employed to treat any of the above conditions, or any other medical condition where electrical stimulation of nerves, muscle, and/or tissue is desired.

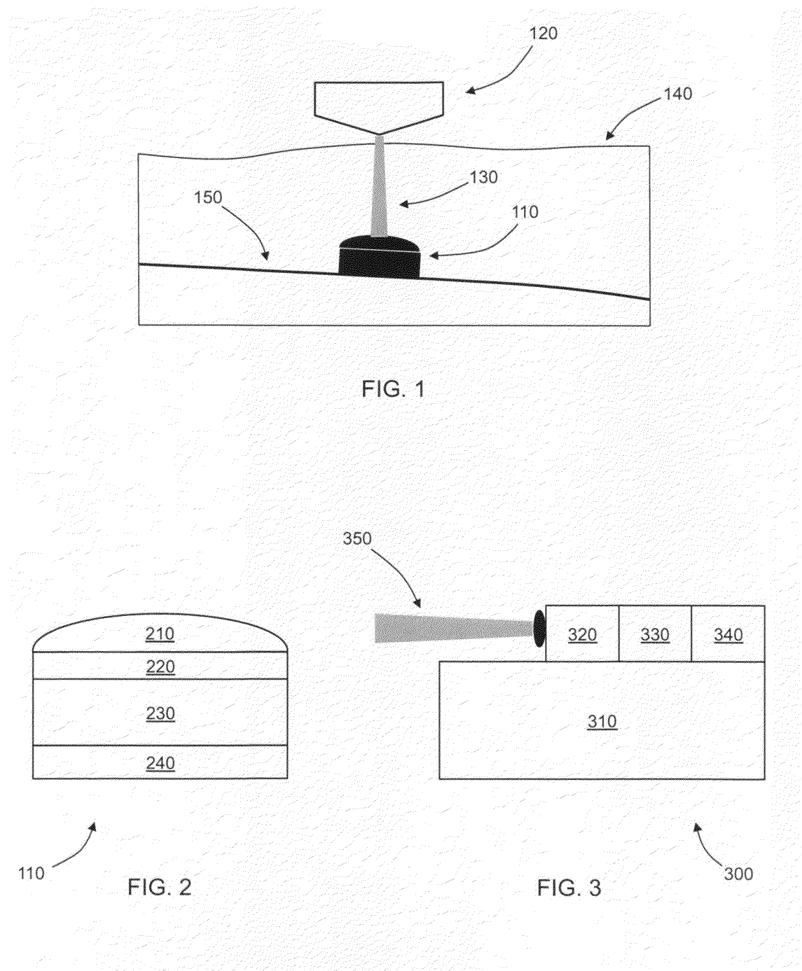

FIG. 1 shows an exemplary embodiment of the present invention with a trigger pod 110 implanted in a subject to provide electrical stimulation to a nerve 150, a muscle, or tissue of the subject. In the embodiment of FIG. 1, an externally located optical transmitter 120 transmits an optical signal 130 through the skin 140 of the subject to the trigger pod 110. In FIG. 1, the optical signal 130 traverses through the body tissue to reach the trigger pod 110 and provide power to it. Once the optical signal 130 reaches the trigger pod 110, the trigger pod 110 applies electrical impulses to the nerve 150. The optical signal 130 can travel through any internal tissues, cavities, fluids, etc., with varying degrees of transmission efficiencies. In certain embodiments, the optical signal 130 is a modulated or un-modulated optical beam with light of infrared or near-infrared wavelengths.

It is noted that in the embodiment of FIG. 1, a line-of-sight configuration is necessary for the optical signal 130 to reach the trigger pod 110. Alignment is necessary to maintain line-of-sight connection between the optical transmitter 120 and the trigger pod 110. In an embodiment, alignment is achieved by monitoring activity of the nerve 150, tissue, or muscle. Alternatively or additionally, alignment is achieved by monitoring a reflection of the optical signal 130 from the implanted trigger pod 110.

An enlarged view of the trigger pod 110 is shown in FIG. 2. The trigger pod 110 includes a micro-power panel 220 to receive optical signals to power the trigger pod 110. In an embodiment, the micro-power panel 220 comprises one or more photodiodes, such as GaAlAs photodiodes, to receive infrared light and convert at least some of the light energy into electrical energy. The electrical energy is used to power the electronic circuit 230, which generates electrical impulses from the electrode 240. Although FIG. 2 shows the electrode 240 at the base of the trigger pod 110, it is noted that the electrode 240 can be placed at any convenient or desired location on the trigger pod 110. It is also noted that the trigger pod 110 can have any shape. In an embodiment, the power conversion efficiency of the micro-power panel 220 is approximately 40% for incident infrared light of approximately 850 nm wavelength. The trigger pod 110 optionally includes a lens 210 to focus the incident optical signal for increased power conversion.

It is important to note that in a preferred embodiment, the trigger pod 110 is battery-less. In other words, the sole power source for the trigger pod 110 is the optical transmitter 120. The absence of an internal battery in the preferred embodiment enables miniaturization of the trigger pods, which reduces the risk of nerve or tissue damage from external mechanical shocks, lowers the chance of infection, and enables easier deployment inside of the body. In an embodiment, each of the trigger pods is less than approximately 7 mm in width. It is noted that in certain embodiments, the electronic circuit 230 includes capacitors for temporary charge storage or small internal batteries for temporary power storage.

The trigger pod 110 is an implantable device that is directly attached to tissue, muscle, or a nerve for neurostimulation. In an embodiment, the trigger pod is implanted at the desired site using a catheter-based process or a mini-surgical procedure. As noted above, the small size of the trigger pod in preferred embodiments allows for easy implantation.

FIG. 3 shows an embodiment of an optical transmitter 300, which can be implanted (either subcutaneously or inside a body cavity of the subject) or located external to the body. Externally located optical transmitters are preferably placed in direct contact with the skin and, in an embodiment, is attached to the skin of the subject using medical adhesives. External optical transmitter can be easily charged or directly connected to a power source, such as a wall outlet or other direct plug-in charge options. An implanted optical transmitter can have a rechargeable or a non-rechargeable battery. In an embodiment, the implanted optical transmitter is recharged using RF charging technology.

FIG. 3 shows an exemplary optical transmitter 300 having a battery 310, and optical instrumentation for generating an optical signal 350. In an embodiment, optical signals are generated with a narrow-band laser diode and the optical instrumentation includes a microcontroller 340, a laser driver 330, and a transmitter optical subassembly 320. In other embodiments, a broader spectrum light-emitting diode (LED) can be used in addition to or in replacement of the laser diode. The optical transmitter 300 can include other components known in the art for generating optical signals 350.

In a preferred embodiment, the optical signal 350 contains optical wavelength light in the near infrared range of 810 nm to 880 nm, with a most preferred wavelength of approximately 850 nm. Near infrared light can penetrate tissue up to about 20 mm thick with acceptable levels of attenuation. The required penetration depth and appropriate wavelength is determined based on the relative position of the optical transmitter and the trigger pod. In an embodiment, the optical signal 350 is a nearly collimated optical beam, which may be modulated or un-modulated.

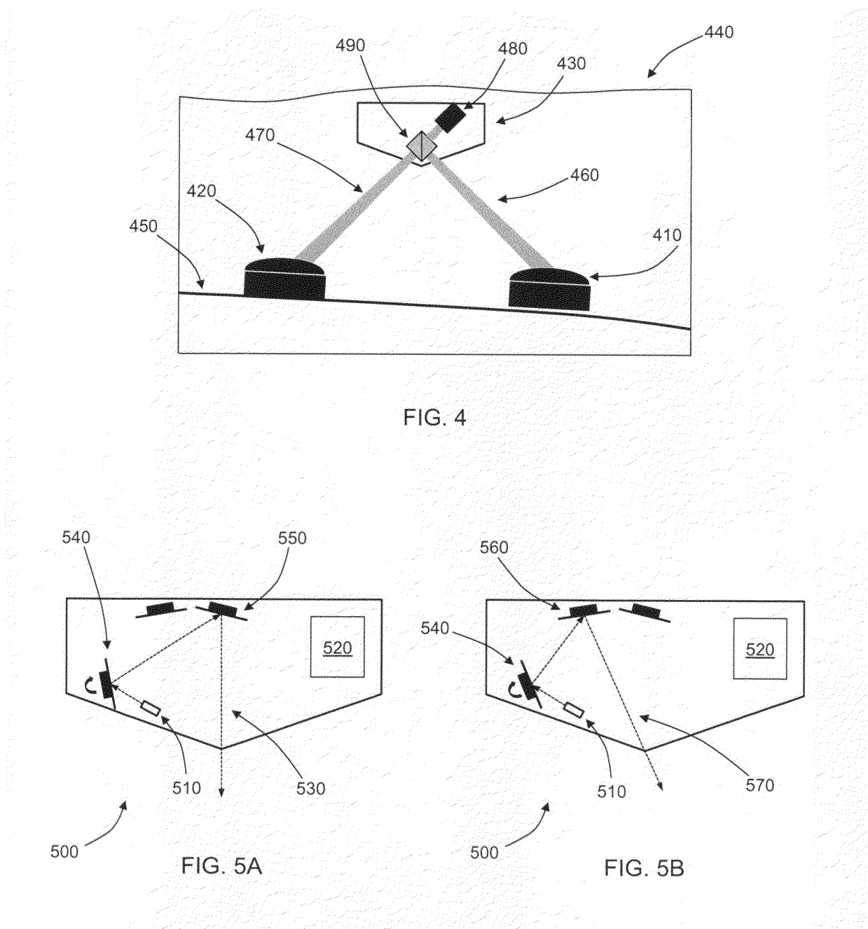

FIGS. 4 and 5A-B show examples of optical transmitters having optical elements such as a beamsplitter or a prism, or a beam steering assembly of micromechanical mirrors to direct the optical signal. In the embodiment shown in FIG. 4, an optical transmitter 430 is implanted under the skin 440 of the subject. The system includes two trigger pods 410, 420 attached to a nerve 450. Both of the trigger pods 410, 420 are optically linked with the optical transmitter 430 through optical signals 460, 470 in a line-of-sight configuration. The source 480 of the optical signals 460, 470 can be a laser diode or a LED.

The optical transmitter 430 includes one or more optical elements 490, such as a beamsplitter or a prism or a pivoted rotatable mirror, used for directing the optical signal to the trigger pods 410, 420. The optical element(s) 490 can be used to direct optical signals 460, 470 to multiple trigger pods 410, 420 simultaneously, or to alternately sweep between multiple trigger pods 410, 420. In a preferred embodiment, the optical element 490 is a pivoted rotatable mirror, with two separate rotational axes (pitch and yaw). By having a pivoted rotatable mirror, the transmission direction of the optical signals 460, 470 can be altered as needed, such as when new trigger pods are introduced or existing trigger pods are moved.

FIGS. 5A-B show an example of an optical transmitter 500 with rotatable micromechanical system (MEMS) mirrors 540-560. In FIGS. 5A-B, the optical source 510 transmits a signal that first reflects off of mirror 540. The orientation of mirror 540 in FIG. 5A directs the optical signal to mirror 550, which reflects the signal to a first direction 530. FIG. 5B shows another orientation of mirror 540, which directs the optical signal to mirror 560, thereby the optical signal is transmitted in a second direction 570. In this way, the optical transmitter 500 can be optically linked with trigger pods located in multiple different locations. The direction of any of the mirrors 540-560 can be controlled using a programmable MEMS controller 520.

Though FIG. 4 only shows two trigger pods 410-420, it is noted that embodiments of the present invention can include any number of trigger pods. Similarly, it is noted that systems of the present invention can include any number of and optical transmitters 430 and is not restricted to systems having only a single optical transmitter.

FIGS. 1, 4, and 6-10 show various embodiments of electrophysiological stimulation systems of the present invention. As would be appreciated by one of ordinary skill in the art, various substitutions, alterations, and deviations from the embodiments shown in these figures could be made without departing from the principles of the present invention. In particular, the present invention includes any combination of any of the systems shown in FIGS. 1, 4, and 6-10.

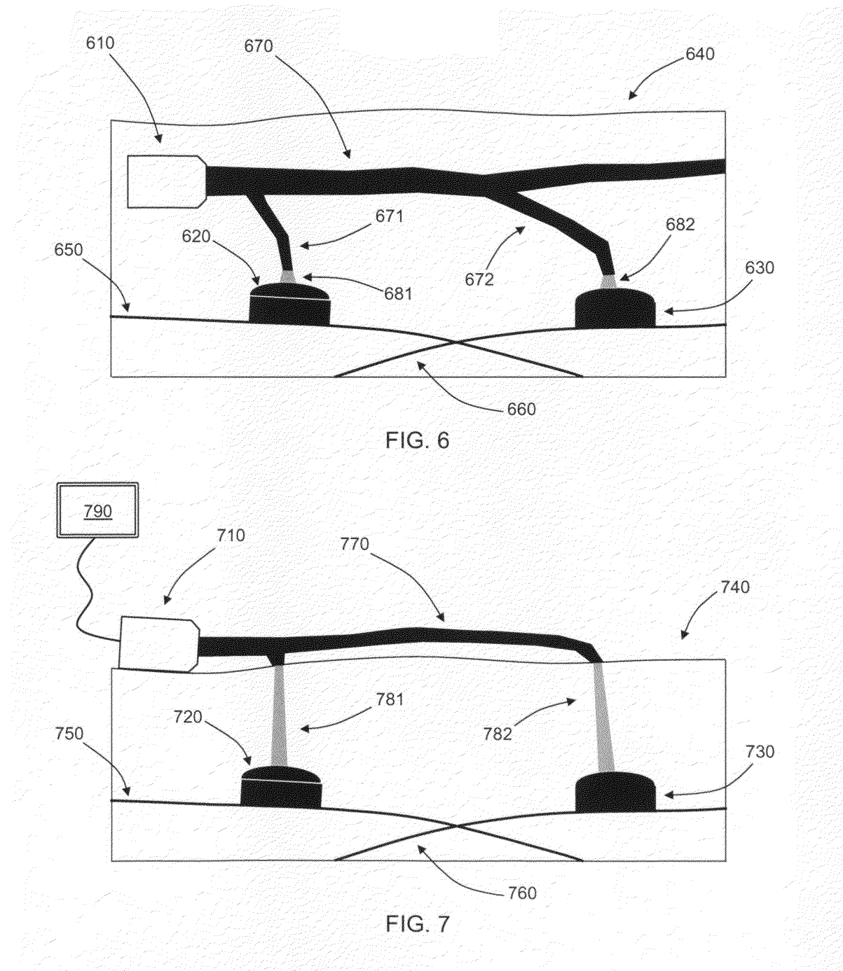

FIG. 6 shows an electrophysiological stimulation system having an optical transmitter 610 and trigger pods 620 and 630 attached to nerves 650 and 660, respectively. The optical transmitter 610 and trigger pods 620, 630 are all implanted under the skin 640 of the subject. The embodiment shown in FIG. 6 also includes a fused multi-furcated optical fiber bundle 670 for directing optical signals 681, 682 to the trigger pods 620, 630 without a line-of-sight requirement. In an embodiment, each of the legs of the optical fiber bundle 670 are positioned such that the end of the leg is proximate to a trigger pod for delivery of optical signals from the optical transmitter 610. For example, leg 671 is pointed at trigger pod 620 and the optical signal 681 is transmitted from the end of leg 671 onto the micro-power panel of trigger pod 620. Similarly, leg 672 delivers optical signal 682 to trigger pod 630. The optical bundle 670 can include any number of optical fibers or legs. Preferably, the number of legs or fibers corresponds with the number of trigger pods.

An embodiment having optical fibers removes the line-of-sight constraint; since the optical fibers can be routed in various ways through or on the body, a line-of-sight configuration is not needed. In an embodiment, the optical fibers or optical fiber bundles have large glass or plastic cores and can have stripped buffers internally to improve packing efficiency. In certain embodiments, a biocompatible polymer buffer surrounds the fiber or bundle externally for protection and durability. Optical fiber diameters range from a few hundred microns to about 3-4 mm and their lengths range from a few inches to a few feet long. In an embodiment, the fiber bundles are routed intravenously or inside of a body cavity.

FIG. 7 shows an embodiment wherein the optical transmitter 710 is located external to the subject. As in the system shown in FIG. 6, the optical transmitter 710 is connected to an optical fiber or a multi-furcated optical fiber bundle 770. However, the fiber bundle 770 is also located externally. In this embodiment, optical signals 781 and 782 are transmitted through the skin 740 to trigger pods 720 and 730, respectively, which generate electrical impulses to stimulate nerves 750 and 760, respectively. In an embodiment, the optical transmitter 710 and the fiber bundle 770 are attached to the skin 740 of the subject using medical adhesives. FIG. 7 also shows a power supply 790 connected to the externally located optical transmitter 710 for powering or recharging the optical transmitter 710.

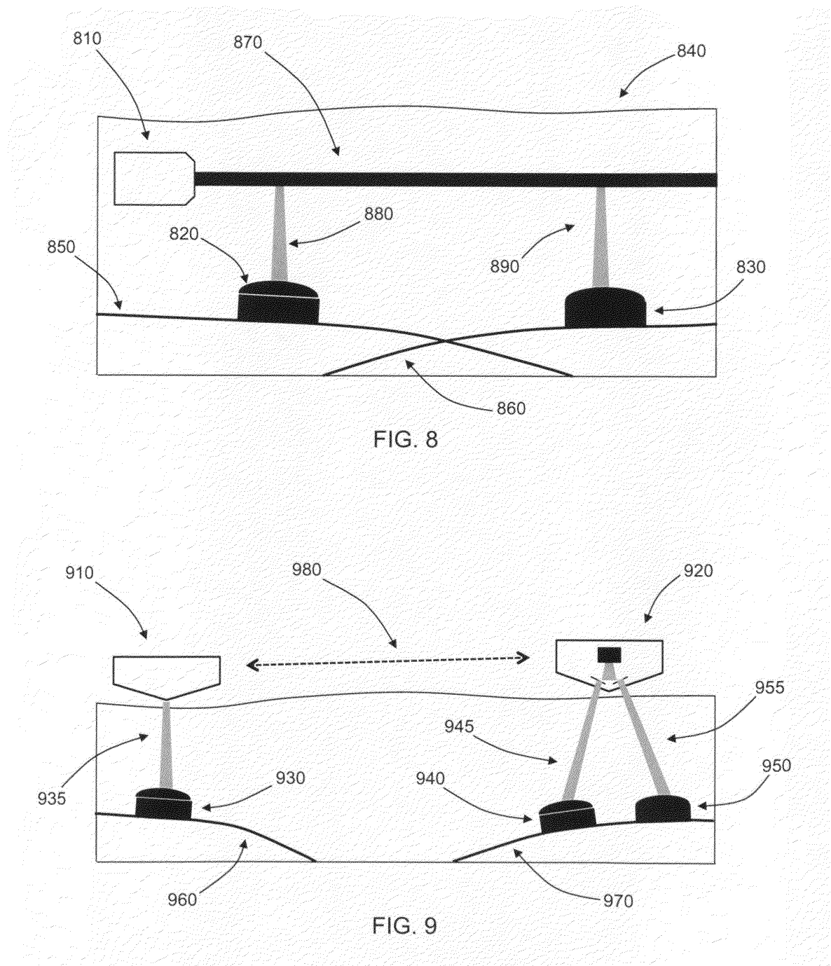

FIG. 8 shows an alternative embodiment having an internally implanted optical transmitter 810, implanted trigger pods 820 and 830 attached to nerves 850 and 860, respectively, and an optical fiber 870, which is also implanted under the skin 840. In the embodiment shown in FIG. 8, the optical fiber 870 has one or more controlled leakage locations along its length, where optical signals 880 and 890 exit the optical fiber 870 and are transmitted to trigger pods 820 and 830. In a preferred embodiment, the optical fiber 876 has a uniform diameter to facilitate routing through the body vessels and cavities.

FIG. 9 shows an electrophysiological stimulation system having multiple optical transmitters 910, 920. Optical transmitter 910 is optically linked through optical signal 935 with trigger pod 930 for stimulating a first nerve 960. Optical transmitter 920 is linked through optical signals 945, 955 with trigger pods 940, 950, both of which are attached to a second nerve 970. Multiple optical transmitters may be required or desired for a variety of reasons, such as if nerves 960 and 970 are placed far apart or in case optical links are difficult to establish. In a preferred embodiment, the multiple optical transmitters 910, 920 are communicatively connected, such as through radio communications 980. Communications between multiple optical transmitters allow for coordinated stimulation by many different trigger pods spaced far apart as may be required in some electrophysiological treatments.

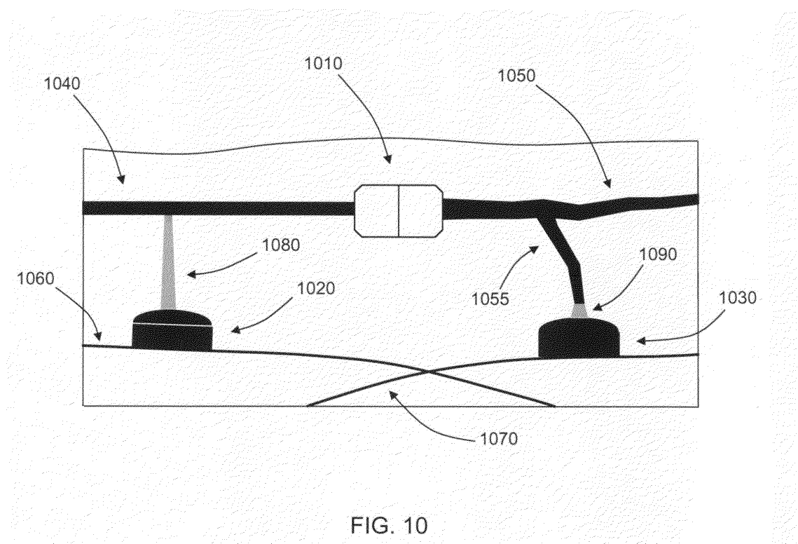

FIG. 10 shows yet another embodiment having an internally implanted optical transmitter 1010, a first trigger pod 1020 attached to a first nerve 1060, a second trigger pod 1030 attached to a second nerve 1070, a uniform width optical fiber 1040 with controlled leakage locations, and a fused multi-furcated optical bundle 1050. An optical signal 1080 is delivered from the leakage location of optical fiber 1040 to trigger pod 1020. A leg 1055 of the multi-furcated optical bundle 1050 is pointed at trigger pod 1030 to deliver optical signal 1090 to trigger pod 1030. It is noted that any number of optical fibers or optical fiber bundles can be used. In an embodiment, a network of optical fibers is present to deliver optical signals to multiple trigger pods.

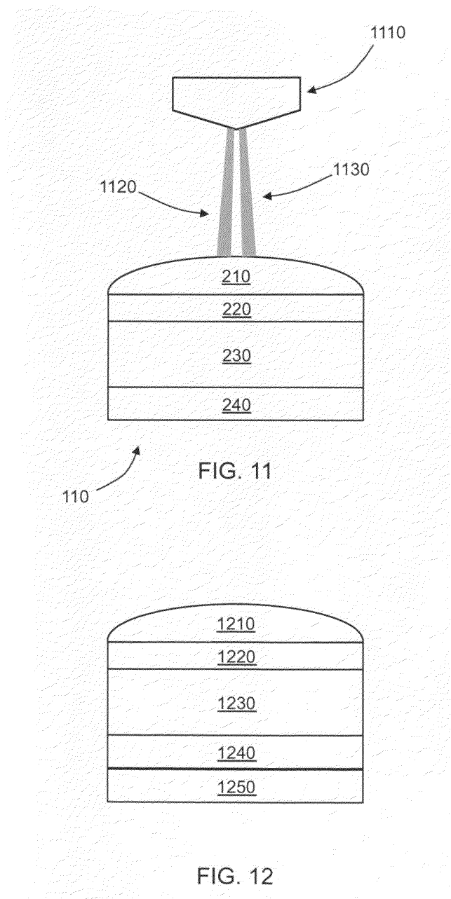

In embodiments of the present invention, such as the embodiments shown in FIGS. 1, 4, and 6-10, the optical signals provide power to the battery-less trigger pods. In certain embodiments, the optical signals can also provide a means for signal/data transmission from the optical transmitter to the trigger pods. In particular, modulation of an optical signal allows for data transfer between transmitter and receiver. FIG. 11 shows an enlarged view of the trigger pod 110 from FIG. 1 receiving a first optical signal 1120 and a second optical signal 1130. In the embodiment of FIG. 11, the first optical signal 1120 is an un-modulated optical beam for power transmission while the second optical signal 1130 is a modulated optical beam for data transmission. The second optical signal 1130 can be used to direct the electronic circuit 230 of trigger pod 110 to control the duration, intensity, timing, or any combination thereof of the electrical impulses delivered by the electrode 240 of trigger pod 110.

In an embodiment, the first (un-modulated) and second (modulated) optical signals can be superimposed or sent simultaneously. Alternatively, power and data transmission can be achieved by transmission of a single modulated optical beam. In this embodiment, the micro-power panel 220 of the trigger pod is capable of converting modulated optical signals into electrical power. Regardless of the nature and types of optical signals, combining power and data transmission allows an operator to have greater control over the electrophysiological stimulation treatment.

FIG. 12 shows another embodiment of a trigger pod. The trigger pod of FIG. 12 includes an input lens 1210, a micro-power panel 1220, an electronic circuit 1230, an energy-harvesting module 1240, and an electrode 1250. In an embodiment, the energy-harvesting module 1240 converts vibrational and/or thermal energy in the environment around the trigger pod into electrical power. The harvested energy can be used in combination or replacement of power from incident optical signals.

As one of ordinary skill in the art will appreciate, various changes, substitutions, and alterations could be made or otherwise implemented without departing from the principles of the present invention, e.g. any number of trigger pods, optical transmitters, and optical fibers can be used, and the components of the system can be either implanted or placed external to the subject. Accordingly, the scope of the invention should be determined by the following claims and their legal equivalents.

What is claimed is:

1. A device for providing electrophysiological stimulation to a subject, said device comprising:
(a) a micro-power panel for receiving a wirelessly transmitted optical signal, wherein said optical signal comprises infrared light, and wherein said micro-power panel converts said infrared light into electrical energy;
(b) an electronic circuit for generating electrical impulses, wherein said electronic circuit is powered by said electrical energy converted by said micro-power panel; and
(c) one or more electrodes, wherein said electrical impulses generated by said electronic circuit are delivered to said subject through said one or more electrodes,
wherein said device is implantable near a muscle, a tissue, or a nerve internal to said subject.

2. The device as set forth in claim 1, wherein said device does not include a battery.

3. The device as set forth in claim 1, further comprising a lens, wherein said lens focuses said optical signal onto said micro-power panel.

4. The device as set forth in claim 1, wherein said micro-power panel comprises one or more photodiodes for converting said infrared light into electrical energy.

5. The device as set forth in claim 1, wherein said optical signal received by said micro-power panel comprises a nearly collimated optical beam.

6. The device as set forth in claim 1, wherein said micro-power panel receives a second optical signal, and wherein said second optical signal comprises data relating to said electrical impulses.

7. The device as set forth in claim 6, wherein said second optical signal comprises a modulated optical beam.

8. The device as set forth in claim 6, wherein said second optical signal directs said electronic circuit to control the intensity, the duration, the timing, or any combination thereof of said electrical impulses.

9. The device as set forth in claim 1, wherein said optical signal comprises a modulated optical beam, wherein said modulated optical beam is converted to electrical energy to power said device, and wherein said modulated optical beam directs said electronic circuit to control the intensity, the duration, the timing, or any combination thereof of said electrical impulses.

10. The device as set forth in claim 1, wherein the width of said device is less than approximately 7 mm.

11. The device as set forth in claim 1, further comprising an energy-harvesting module, wherein said energy-harvesting module uses vibrational energy or thermal energy to power said device.

12. A wireless system for providing electrophysiological stimulation to a subject, said system comprising:
(a) an optical transmitter for transmitting optical signals; and
(b) one or more implantable trigger pods, wherein each of said trigger pods comprise:
(i) a micro-power panel for receiving said optical signals transmitted by said optical transmitter, wherein said micro-power panel converts said optical signals into electrical energy;
(ii) an electronic circuit for generating electrical impulses, wherein said electronic circuit is powered by said electrical energy converted by said micro-power panel; and
(iii) one or more electrodes, wherein said electrical impulses generated by said electronic circuit are delivered to said subject through said one or more electrodes,
wherein said one or more trigger pods are implanted near a muscle, a tissue, or a nerve internal to said subject, and wherein said one or more trigger pods are wirelessly connected to said optical transmitter.

13. The system as set forth in claim 12, wherein each of said implantable trigger pods does not include a battery.

14. The system as set forth in claim 12, wherein said optical transmitter comprises a laser diode or a light-emitting diode, and wherein said laser diode or said light-emitting diode produces said optical signals transmitted by said optical transmitter to said trigger pods.

15. The system as set forth in claim 12, wherein said optical transmitter comprises one or more optical elements, wherein said optical elements comprise a beamsplitter, a prism, a mirror, or any combination thereof, and wherein said one or more optical elements directs said optical signals from said optical transmitter to said trigger pods.

16. The system as set forth in claim 15, wherein one of said optical elements is a pivoted rotatable mirror, and wherein said pivoted rotatable mirror rotates to direct said optical signals to two or more of said trigger pods.

17. The system as set forth in claim 12, wherein said optical transmitter is implanted in the body of said subject.

18. The system as set forth in claim 12, wherein said optical transmitter transmits a second optical signal, wherein said micro-power panel of one of said trigger pods receives said second optical signal, and wherein said second optical signal directs said electronic circuit of the same of said trigger pods to control the intensity, the duration, the timing, or any combination thereof of said electrical impulses delivered by the same of said trigger pods.

19. The system as set forth in claim 12, further comprising one or more optical fibers wherein said optical transmitter transmits said optical signals to said trigger pods through said optical fibers.

20. The system as set forth in claim 19, wherein one or more of said optical fibers is implanted in the body of said subject.

21. The system as set forth in claim 19, further comprising two or more of said trigger pods, wherein each of said optical fibers corresponds with one of said trigger pods, and wherein the ends of each of said optical fibers are located proximate to said micro-power panel of said corresponding trigger pod.

22. The system as set forth in claim 19, wherein said optical transmitter and said optical fibers are located external to the body of said subject, and wherein said optical signals are delivered through the skin of said subject to said trigger pods.

23. The system as set forth in claim 12, further comprising a multi-furcated fused fiber bundle, wherein said optical transmitter transmits said optical signals to said trigger pods through the legs of said multi-furcated fused fiber bundle.

24. The system as set forth in claim 12, further comprising an optical fiber having one or more leakage locations, wherein said optical signals are delivered from said optical transmitter to said optical, and wherein said optical signals exit said optical fiber through said leakage locations.

25. The system as set forth in claim 12, further comprising multiple optical transmitters, wherein each of said optical transmitters transmits said optical signals to one or more of said trigger pods.

26. The system as set forth in claim 25, wherein at least two of said multiple optical transmitters are communicatively connected.

27. A method of providing electrophysiological stimulation to a subject, said method comprising:
(a) providing an optical transmitter for transmitting optical signals; and
(b) implanting one or more trigger pods near a muscle, a tissue, or a nerve of said subject, wherein each of said trigger pods comprises:
(i) a micro-power panel for receiving said optical signals transmitted by said optical transmitter, wherein said micro-power panel converts said optical signal into electrical energy;
(ii) an electronic circuit for generating electrical impulses, wherein said electronic circuit is powered by said electrical energy converted by said micro-power panel; and
(iii) one or more electrodes, wherein said electrical impulses generated by said electronic circuit are delivered to said subject through said one or more electrodes; and
(c) directing said optical transmitter to transmit said optical signals to said trigger pods, whereby said electrical impulses provide electrophysiological stimulation to the muscle, the tissue, or the nerve of said subject.

28. The method as set forth in claim 27, further comprising directing said optical transmitter to transmit a second optical signal to said trigger pods, wherein said micro-power panel of one of said trigger pods receives said second optical signal, and wherein said second optical signal directs said electronic circuit of the same of said trigger pods to control the intensity, the duration, the timing, or any combination thereof of said electrical impulses delivered by the same of said trigger pods.

29. The method as set forth in claim 27, wherein at least one of said trigger pods is implanted near the heart of said subject, and wherein said electrical impulses delivered by the same of said trigger pods are for treating arrhythmia.

30. The method as set forth in claim 27, wherein at least one of said trigger pods is implanted near one of the nerves of said subject, and wherein said electrical impulses delivered by the same of said trigger pods are for providing pain relief to said subject.
